(19) United States
(12) Patent Application Publication
(10) Pub. No.: US 2003/0230308 A1
(43) Pub. Date: Dec. 18, 2003

(54) Medical ventilator with a graphics interface allowing designation of target values

(75) Inventor: Dan Linden, Stockholm (SE)

Correspondence Address: SCHIFF HARDIN & WAITE, 6600 SEARS TOWER, 233 S WACKER DR, CHICAGO, IL, 60606-6473, US

(73) Assignee: Siemens Elema AB

(21) Appl. No.: 10/459,279

(22) Filed: Jun. 11, 2003

(30) Foreign Application Priority Data
Jun. 18, 2002 (SE) 0201854-7

(51) Int. Cl. A61M 16/00

(52) U.S. Cl. 128/204.18; 128/204.21

(57) ABSTRACT

A medical ventilator has a user interface with a screen adapted to display curves representing function parameters for the medical ventilator, a memory unit for storing target values for the function parameters, and a control unit for controlling the medical ventilator dependent on the target values for the function parameters stored in the memory unit. A simplified method of modifying or programming the medical ventilator is achieved through adaptation of the user interface to register a new curve and/or changes to a displayed curve performed by a user on the screen and to transfer these to the memory unit for storage as new target values for the function parameters.

### BACKGROUND OF THE INVENTION

1. Field of the Invention

The present invention relates to a medical ventilator of the type having a user interface with a screen on which curves are displayed representing function parameters for the ventilator, and having a memory for storing target values for those function parameter, and a control unit for controlling the ventilator dependent on the stored target values.

2. Description of the Prior Art

The user interface is an important component of a medical ventilator. It normally includes a screen that can be used to display numerical and graphical information related to operating parameters, ventilation modes, monitored parameters, respiration curves, etc. One such interface is described in U.S. Pat. No. 5,881,723.

It is also known to provide a medical ventilator with a user interface having an interactive screen. An example of such is the Servoⁱ® ventilator from Siemens Elema AB, Sweden. This user interface has an interactive screen that selectively can be used for programming of functions and as a monitor to display breathing curves and other information.

In the present context programming of functions means chiefly breathing modes, wherein the parameter values can be entered and numerically displayed on a screen.

In certain situations it would be desirable to enter or modify a breathing mode outside the normal input possibilities. This should be possible to perform in a simple and user-friendly fashion, while still being accurate and precise.

U.S. Pat. No. 5,931,160 describes a control system for ventilators, which allows an essentially free setting of different breathing parameters. The setting is achieved via usual installation methods. The input values can be displayed on a screen. This programming requires the user to enter a whole range of parameters, successively leading to a ventilation mode.

### SUMMARY OF THE INVENTION

An object of the present invention is to provide a medical ventilator of the type described above which at least alleviates the above problems and at least partly satisfies the above-described desires.

The above object is achieved in accordance in accordance with the principles of the present invention in a medical ventilator of the type initially described, wherein the user interface allows a user to designate a substitute curve on the screen, which may be a new curve and/or changes to a previously-displayed curve, with the substitute curve being transferred to the memory unit for storage therein as new target values for the function parameters.

With a user interface, designed to register desired settings or changes to settings, a very simple and direct work tool is obtained for the operator. No buttons need be pressed. A new curve shape, for example the pressure, can quickly and simply be drawn in by the operator and implemented by the medical ventilator.

To avoid unintentional changes of the installed values the interactive screen can be designed so that a switch must be adjusted before changes can be implemented.

Restoration of the last (fixed) installed operational mode can be done by means of another switch.

### DESCRIPTION OF THE DRAWINGS

FIG. 1 is a schematic representation of an exemplary embodiment of a medical ventilator according to the invention.

FIG. 2 is an interactive screen in the medical ventilator according to FIG. 1.

FIG. 3 is an alternative arrangement of a screen in the medical ventilator according to the invention.

### DESCRIPTION OF THE PREFERRED EMBODIMENTS

FIG: 1 shows an exemplary embodiment of a medical ventilator 2 according to the invention. The medical ventilator 2 has a pneumatic unit 4 for the preparation of a breathing gas. In the present case the pneumatic unit 4 has two gas inlets 6A, 6B for coupling in of two gases, for example oxygen and air.

The prepared breathing gas is carried toward a patient 8 via an inspiration line 10 during inspiration and away from the patient 8 via an expiration line 12 during expiration.

The medical ventilator 2 further has a control unit 14 for regulation and control of the pneumatic unit 4 and a user interface 16, through which an operator can enter a suitable ventilation mode for the patient 8.

The user interface 16 in this embodiment has an interactive screen 18. To increase safety against unwanted changes or settings, a first function switch 20A may be included. Interactive measures between the operator and screen 18 would then be permitted only after activation of the first function switch 20A.

To facilitate the resetting of the last programmed setting, the user interface 16 may include a second function switch 20B. Upon activation of the second function switch 20B the last used setting (for controlling supply of gas to a patient) is restored.

The function of switches 20A, 20B of course can be implemented in a single switch. The switches 20A, 20B can be hardware switches located next to or on the interactive screen 18, software based switches displayed on the screen, or a mixture of the two.

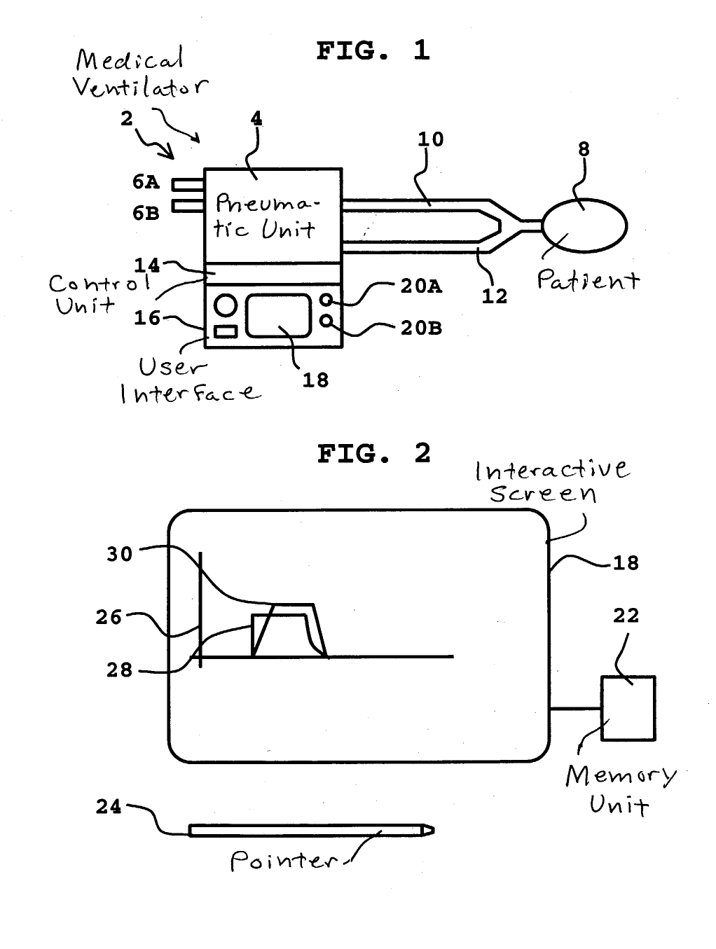

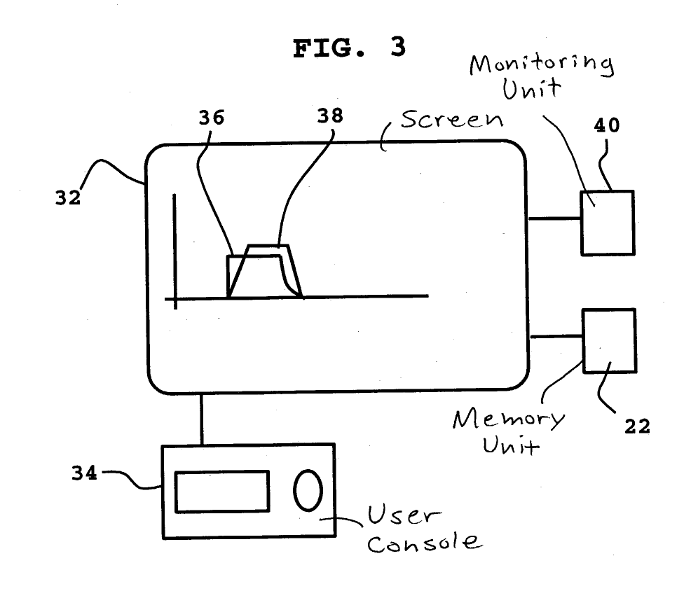

In FIG. 2 the interactive screen 18 is shown more clearly. A memory unit 22, which is here connected to the screen 18, is also shown. The memory unit 22 is also connected to the control unit 14 in FIG. 1 (not shown in FIG. 2).

The function parameters for the ventilation mode which shall be applied to the patient 8 are found in the memory unit 22. More specifically there is to be found, among other things, target values for one or more of the parameters: pressure, flow, inspiration time and expiration time. Other parameters may also be found, such as composition of the breathing gas, etc.

The interactive screen 18 of the foregoing exemplary embodiment can be modified by means of a pointer device 24. The pointer device 24 is not essential but does allow a more precise revision of the screen contents than does the use of a finger.

A coordinate system 26 is drawn on the screen 18 as an example of a graphic representation of the actual ventilation mode (corresponding target values in memory unit 22). A first curve 28 is displayed in the coordinate system 26, which corresponds to a pre-programmed working mode (pressure on the vertical axis and time on the horizontal axis) over a breathing cycle (inspiration and expiration).

In accordance with the invention the operator can modify the existing ventilation mode (alternatively create his or her own) by changing the first curve 28 to the second curve 30 directly on the screen 18. Values for the second curve 30 are then registered in the memory unit 22 as target values and transferred to the control unit 14 for the control of the pneumatic unit 4 according to the modified ventilation mode.

In order to facilitate the setting for the user, a numerical value could be displayed indicating the value of the touched point on the screen. The value could be indicated in a box that follows the movements made by the user on the screen.

The new input value can be implemented as the pointer device 24 is lifted from the screen or via a confirmation step.

If an entirely individualized ventilation mode is to be created the coordinate system can be blank.

FIG. 3 shows another embodiment with a screen 32. The screen 32 is not interactive and inputs are made through a user console 34, which could be a keyboard, mouse or any other device for entering data (alone or in combination). A mouse or similar device may be preferable as they provide a more easily movable cursor or indicator on the screen. All known means of transferring the information by wire or wireless can be utilized.

The purpose is still to create a curve on the screen 32, either by drawing the entire curve or supplementing key points that are then combined into a curve by the user interface (interpolation).

As an alternative to proceeding from a blank screen or from target values to an existing programmed ventilation mode, a measured curve 36 can be shown on the screen 18 and modified in a corresponding way to a modified curve 38. The measured curve can be taken from a monitoring unit 40. This essentially means that a measured curve 36 showing what happens near or in the lungs of a patient can be used as basis for modifications of a treatment.

Thus the intended or desired effect of a certain ventilation mode can be achieved in an even more effective manner. The registration of curves for pressure, flow and volume is made, as a rule, in the majority of medical ventilators. In the event of a certain parameter or variable being missing from the medical ventilator's normal monitoring features then a corresponding separate parameter meter can simply be connected in order to attain a relevant measurement signal.

The actual changes to/insertions of curves on the screen 32 can be achieved in many different ways as understood from the above description in relation to FIG. 2 and FIG. 3. One way is for the operator to draw in the complete curve and another is for the operator to insert key points, after which the user interface adapts a curve to the inserted points.

Naturally, combinations in functionality and hardware/software can be made between the two embodiments of FIG. 2 and FIG. 3. Typically, monitored curves as shown in FIG. 3 could also be utilized in the embodiment of FIG. 2.

Further safety functions, of course, also can be utilized for the disclosed manner of input. There will then be no risk for the patient of being exposed to pressures and/or volumes that are too large, unreasonably short or long inspiration and expiration times, etc. Basically, any known safety function relating to the overall operation of the medical ventilator vis-à-vis the patient can be used.

All breathing apparatuses for medical use are included in the context of medical ventilator used in the present application. Accordingly, respirators or ventilators for intensive care, anaesthetic apparatus, respirators or ventilators for sub-acute, respirators for home care, etc., are included.

Although modifications and changes may be suggested by those skilled in the art, it is the intention of the inventor to embody within the patent warranted hereon all changes and modifications as reasonably and properly come within the scope of his contribution to the art.

I claim as my invention:

1. A medical ventilator system comprising:
a ventilator arrangement adapted for interaction with a patient;
a user interface having a display screen on which curves are displayed representing function parameters for said ventilator arrangement;
a memory in which target values for said function parameters are stored;
a control unit connected to said ventilator arrangement for controlling the ventilator arrangement dependent on the target values stored in said memory; and
said user interface having an input arrangement allowing a user to designate, on said screen, a substitute curve for at least one of said function parameters, selected from the group consisting of a new curve and changes to a displayed curve, said user interface transferring said substitute curve to said memory for storage therein as new target values for said at least one of said function parameters.

2. A ventilator system as claimed in claim 1 wherein said input arrangement allows a user to enter designated points on said screen, and wherein said user interface generates said substitute curve by interpolation between said points.

3. A ventilator system as claimed in claim 1 wherein said user interface comprises a function switch which must be activated to allow said designation of said substitute curve.

4. A ventilator as claimed in claim 3 wherein said function switch is a first function switch, and wherein said user interface comprises a second function switch which, when activated, automatically recovers previously stored target values from said memory.

5. A ventilator system as claimed in claim 1 wherein said screen is an interactive screen, and forms said input unit.

6. A ventilator system as claimed in claim 5 wherein said substitute curve is a curve representing changes to a displayed curve, and wherein said ventilator system comprises a stand-off alone pointer, manipulatable by the user, allowing the user to designate said changes on a curve displayed on said interactive screen.
